(19) United States
(12) Patent Application Publication
(10) Pub. No.: US 2018/0340202 A1
(43) Pub. Date: Nov. 29, 2018

(54) Method for Rapid High Volume Production of Synthetic Vaccines

(71) Applicant: Richard Postrel, Miami Beach, FL (US)

(72) Inventor: Richard Postrel, Miami Beach, FL (US)

(21) Appl. No.: 15/719,322

(22) Filed: Sep. 28, 2017

Related U.S. Application Data
(60) Provisional application No. 62/510,268, filed on May 23, 2017.
(60) Provisional application No. 62/510,442, filed on May 24, 2017.

(51) Int. Cl. C12P 21/00; C07K 14/005; C12M 1/40; C12M 1/12; C12M 3/00

(52) U.S. Cl. CPC C12P 21/00; C07K 14/005; C12M 23/44; C12M 25/00; C12M 21/18

(57) ABSTRACT

The present invention features an on demand programmable machine and process for producing synthetic vaccine or other protein product(s). This system includes modular machine components with programmable inputs to responsively and rapidly synthesize selected protein product(s) in desired amounts. When the device is programmed to produce multiple proteins in a single run, the amount of each protein produced is independently controlled. The programmability and modularity is scalable permitting e.g., low volume output to manage an incipient local outbreak, and in cases where higher production volumes are necessary to prevent epidemic or pandemic events, high levels of production can be planned at the outset or scaled up as greater need is recognized.

The US Centers for Disease Control Recommends annual flu vaccination for everyone older than six months. Delivering this presents an annual logistics exercise because flu viruses are continuously changing and thus require frequent update of the flu vaccine formulation to remain effective. Every dose of the flu vaccine contains products from three or four flu viruses, generally at least one from each of type A and type B subtypes recognized as current versions of the disease. There are two types of changes which are constantly monitored by the CDC, the World Health Organization and in influenza centers of more than 100 countries.

Antigenic drifts are small changes in the viral genome that only minorly impact antigenicity (the process of being recognized by our immune systems) in the short term, but over time may change enough so that increasing numbers of vaccinated people will no longer mount an adequate antigenic response. These drifts in both types A and B are a major factor driving annual flu vaccine revisions.

However, type A viruses can change abruptly in a process called antigenic shift. This is a major change in the viral proteins producing a viral particle so different from other versions that few people have immunity and the virus can rapidly spread. The type A viruses shift by exchanging large amounts of their genetic code with other viral subtypes even from viruses that infect non-human species. This results in major outbreaks or epidemics such as the avian flu. These zoonotic viral shifts if not quickly addressed often result in widespread epidemics and pandemics.

Since the make-up of the circulating virus is constantly changing, the CDC monitors circulating viruses throughout the year and provides new and updated information about the vaccine match as it becomes available. Each spring the US Food and Drug Administration directs which vaccine viruses are to be used for the flu vaccine.

One important practical factor in the FDA recommendation of which viruses to include in the annual flu vaccine is whether or not there is a good vaccine virus available; that is, a virus that could be used in vaccine production and which would likely protect against the viruses likely to circulate during the upcoming season. Vaccine viruses that infect humans must also be able to grow in chicken eggs. They need to be tested and available in time to allow for production of the large amount of vaccine virus needed to make vaccine for fall distribution. Sometimes, a suitable vaccine virus cannot be isolated and developed (for growth in eggs) in time to be included in the upcoming season's vaccine. For example, some influenza viruses, like H3N2 viruses, grow poorly in eggs, making it difficult to obtain candidate vaccine viruses. And in some years one or more influenza viruses may not appear and spread until late in the influenza season, making it difficult to prepare a candidate vaccine virus in time for vaccine production.

To address at least the egg compatibility impediment, in 2016 the FDA approved using cultured mammalian cells for some vaccine productions. This appears to be an improvement over insect cell culture that had been implemented in an effort to provide vaccines for those allergic to eggs.

The present invention improves these production methods by eliminating the egg and/or the proliferating cell as the factories for producing intact viral particles for processing into vaccine. The steps of adapting the viruses for growth in cells or eggs, the requirement for a supply of healthy eggs, addressing local drifts and shifts are avoided. The on-demand system reduces the delay or lead time necessary for conventional production methods and because these steps are eliminated is capable of small or large scale production. The simplified system for vaccine production enables the vaccine to be optimized for delivery to the specific location when most effective, i.e., at the earliest sign of an outbreak.

Forms of vaccinations have a long history. But in contrast to the continuous need for change as exemplified in the annual flu vaccination formulation, many pathogenic organisms have maintained a consistent genome allowing vaccines to protect against these pathogens for several years, perhaps even a lifetime post vaccination. Current CDC scheduling recommends vaccinations for over 20 stable pathogens, including, but not limited to: measles, mumps, rubella, rotavirus, hepatitis a, hepatitis B, Dtap (diphtheria, tetanus and acellular pertussis) inactivated polio, live polio, varicella, mennigoccal ACWY, Ttap (tetanus, diphtheria and acellular pertussis), HPV, meningoccal B, pneumoccal polysaccharide, etc. Vaccinia (smallpox) vaccinations are no longer recommended since the disease has been eliminated worldwide. However, the United States and other countries maintain dispersed stockpiles of smallpox vaccine, should an outbreak occur.

The present invention, while especially available for rapid response to shifted pathogens or to new outbreaks is also available as a production means for maintaining steady stream supply lines and/or to produce and maintain back-up vaccinations for storage.

### BACKGROUND

### Disease Contagion

Although infectious diseases were not recognized as “germs” in the sense we understand contagion today, infecting humans with a bit of biologic material harvested from disease abscess has a history extending back over a thousand years. In the early practice, a small dose of the disease (scratch from a pustule) was used to inoculate a person with the hope that a small case of disease would forestall a major disease event in the future. Fatal reactions were common, but benefits versus risks though controversial were acceptable to many societies.

Smallpox was a worldwide disease with 20 to 40 percent of an infected population dying from the infection. In Asia there is evidence that live vaccine harvested from pustules of humans infected with smallpox were scratched under the skin as a form of vaccination. Though frequently fatal, fatalities seemed less than in those spuriously infected.

Edward Jenner is credited with the concept of using an attenuated virus for vaccination. The vaccine he used was harvested from pox pustules on a diseased cow. The cowpox virus produced a mild form of pox disease in humans, but notably forestalled future infection from the human form of pox virus, smallpox. This early vaccine was introduced by breaking the skin using a sharp object that had scraped pustule material from a donor source. At first the vaccine was obtained from cow pustules, but soon distribution was from one human cowpox pustule to anther human who could then serve as a vaccine source. But vaccine was only available for a few days following infection before the immune system resolved the disease and pustules were eliminated. This practice also was responsible for spreading other diseases that the pustule source person might be carrying, for example, syphilis. The need for a controlled source of vaccination material was recognized.

An important aspect of the understanding of the vaccination process and the various sources of immunogen used for vaccination was the acceptance of germ theory, the belief that microbial beings caused disease by propagating in tissues of the infected person or animal. Once germ theory gained general acceptance, vaccination/immunization gained popular support.

### Immune System and Immunization

Immunization campaigns to prevent or eliminate a disease by intentionally and artificially creating immunity by inoculation with a “practice” substance so that the body learns to protect itself from the illness of concern took off in the early twentieth century.

We now understand that this “practice” substance is an antigen that elicits an immune response that can attack a disease before it causes serious harm in the infected organism. The “practice” antigen has had many formats as vaccination practice has developed. We would refer to the intact cowpox virus today as a live attenuated virus, that is - a weak virus that propagates in an individual without serious harm, but that then protects the individual from a more serious infection.

Live-attenuated polio vaccine (the Sabin vaccine) has played a large role in the near elimination of polio, but this live vaccine has the miniscule—though real-danger of infecting those rare individuals with compromised (defective) immune systems. Public health benefits from a concept known as herd immunity, which is when a sufficiently large fraction of the population is immune, thus preventing, by reducing to negligible, transmission of the virus should one individual become infected. When the probability of a sporadically infected individual coming in contact with a susceptible individual approaches zero, the probability of a second individual being infected likewise becomes zero.

In this regard, one advantage of live attenuated virus is that (especially in the case of the Sabin vaccine) attenuated infection can spread to non-inoculated individuals (e.g., for Sabin vaccine: care givers changing diapers of a vaccinated infant). In contrast, a killed vaccine, such as the Salk polio vaccine does not and cannot cause disease even in immune compromised individuals since the virus is “dead”, but this mode of vaccination does not contribute to spreading herd immunity through shared infection. A danger with the killed vaccine approach is that it uses a live vaccine in production and improper/incomplete killing can thereby cause, rather than prevent, disease.

Recent versions of flu vaccine have been approved and administered in both live attenuated and killed virus formats. Although inoculated persons usually retain some immunity for several years following inoculation, antigenic drift—minor change of the epitope in the subtype that is caused by point mutations in one or more epitopic genes—may result in a local or widespread epidemic: since many or most of the immunized persons have failed to develop antibodies against something to which they were never exposed. However, some immunity may arise from shared or similar epitopes common to the related members in subtypes.

More serious for rapid spread of disease is an antigenic shift in the pathogen—from a major change of epitope. The shift is characterized as a new subtype, usually the result of introduction of a gene or major fragment from another virus. Such shifts can result in a pandemic since little immunity exists in the relevant population.

Live attenuated viral vaccines have their own particular difficulties and risks. When immunogenicity is closely related to genes required for growth or infection, attenuation may not be possible. Selective pressures on attenuated viruses may cause rapid drifts to less immunogenic gene expressions or less compatibility with the vaccine targeted virus. Viruses can be difficult to culture. If attenuation impedes culture still further, adequate supply may be resource intensive, expensive or impossible. And, an attenuated virus may mutate or may swap a gene with another virus in the vaccinated individual to restore its virulence. This has been observed with at least on strain of live attenuated polio vaccine. An in vitro vaccine system as used in practicing the present invention avoids the concerns relate to killed/inactivated viruses and live attenuated viruses.

Vaccines are complex mixtures that comprise the active immunizing agents, but also other potentially allergenic substances. To date, the most frequently reported vaccine allergy events are to MMR vaccine, a live attenuated variety. Initially these reactions were attributed to egg protein. But the host cell producers infect to culture the virus is chick embryo fibroblasts (not egg) and contains negligible egg protein. The vaccine has been safely administered to children allergic to eggs making egg allergy an unlikely cause of reactions to the vaccine. A report implicated gelatin, a stabilizing component as the probable allergen responsible for anaphylaxis. Allergic reactions to varicella vaccine also have been attributed to this gelatin.

Two common vaccines, those for influenza and Yellow Fever, use egg hosts; patients allergic to eggs have presented with severe allergic reactions. In those many other instances that are not attributable to the virus' host, allergic reactions will not be directly attributable to the pathogenic antigen, because: for such allergic response, there must have been previous exposure to produce the antibodies that cause the reactive shock. These must be induced by a previous contact with the recognized antigen.

Since many viruses have several commonalities with random other viruses, exposure to another pathogen with similar epitope is possible (and even likely given enough multiple exposures). The later allergic responses to the vaccine immunogen(s) itself cannot be ruled out. Simplified compositions using proven excipients and selected proteins, rather than degraded virus particles, as vaccine antigens can lessen potential for allergic responses. Careful selection of antigenic proteins to be used in a composite protein vaccine is one means of reducing such random anaphylactic concern.

To activate the immune system and produce antibodies and reactive T cells the antigenic proteins undergo processing and presentation where MHC antigen-presenting cells express antigen on their cell surface in a form recognizable by lymphocytes. Antigen processing includes protein fragmentation (proteolysis), association of the fragments with MHC (major histocompatibility complex), and expression of the peptide-MHC complex at the cell surface where they can be recognized by the T cell receptor (TCR).

There are two classes of MHC molecules, MHC class I and MHC class II.

MHC class I molecules present endogenously synthesized proteins, including both self-proteins and viral proteins synthesized by infected host cells. The complexes are presented to TCR on CD8⁺ T-cells. These intracellular proteins become degraded into antigenic peptides by the 26S proteasome and are transported into endoplasmic reticulum (ER) via TAP1 and TAP2 transporters. TAP-binding protein, tapasin, bridges MHC class I molecules to TAP transporters. The MHC class I heavy chains bind the ER chaperone calnexin. After folding, the heavy chains dissociate from calnexin and are free to bind beta-2 microglobulin, ER chaperone calreticulin and thiol oxodoreductase PDIA3. Then, MHC class I proteins bind antigenic peptides in their cleft for presentation. The MHC class I-peptide complex is transported to the plasma membrane for presentation to CD8⁺ T-cells. MHC class I heavy chains that fail to acquire a mature conformation in ER as a result of defective folding or assembly are degraded in the proteasome.

MHC class II proteins are responsible for presenting exogenous antigens to TCR on CD4⁺ T-cells. In the process, the exogenous proteins are endocytosed and degraded by acidic proteases (e.g., by legumain, cathepsin L and cathepsin S) in endosomes and lysosomes. γ-interferon-inducible lysosomal thiol reductase IP-30 cleaves S—S bonds to help unfold endocytosed antigens transported to MHC class II-containing compartments. The MHC class II molecules are assembled in the ER by the chaperone, CD74, which also directs the complexes to the endosomal compartments. CD74 processing includes cleavage by legumain, then by cathepsin S and Cathepsin L. Finally, HLA-DM, a vesicle membrane protein, mediates CD74 removal from MHC class II polypeptides. After this, MHC class II molecules bind antigens to form a complex. MHC class II/antigen complex is then transported to cell surface for presentation to CD4⁺ T-cells. In some situations, e.g., where the MHC types can be matched to the recipient(s), the complex is more adept at creating an immune response. Since the foreign peptide source material and/or complex comprise proteins/polypeptides the present invention is suitable for production.

### SUMMARY OF THE INVENTION

One vaccination approach involves assembling a collection of proteins that self-assemble to mimic to a degree the outer surface of the disease organism, the portion of the intact pathogen initially exposed to the immune system. These proteins can be artificially produced so that no disease organism needs to be eliminated and the proteins themselves can produce strong immunogenic responses. Covalent cross links can be added to improve stability of three dimensional structure. A “right” mix of proteins (sometimes genetically modified) can take years for manufacture of candidates and the required research to find, but the strong and often lasting immune response makes these particularly effective and able to garner high prices per dose. Degradation by proteasomal proteolytic proteins or even isolated proteasomes can be used to select presentation fragments suitable for MHC1 presentation.

A less vigorous but still effective technique uses antigenic protein or a collection of proteins that do not self-assemble into disease like particles. When properly configured, these proteins may be taken up by immune cells and presented for establishing an effective immune response that protects against future infection. Molecular biology has allowed growth of such proteins in cultures of bacteria or yeasts.

Early vaccines required an infected animal or human to culture virus that could be harvested from that individual for vaccinating others. In early vaccination history, this was a low cost method because, for example in Jenner's work, cows and milkmaids were naturally infected by the cowpox virus and the diseased pustules were readily harvestable for sharing with the vaccine recipient(s).

But as understanding of germ theory progressed and vaccines began to require killed, fragmented or specified epitopes, vaccine production became industrialized in the sense that selected organisms were kept or grown as factories to produce the materials for vaccination. These life forms serving as factories to culture from which vaccine materials were extracted needed to be bred, grown and fed.

Historically, plants and animals have been a source of elements and energy we use to sustain life. Although plants used for food production can provide easy harvest they are often extremely inefficient. For example, the apple requires a tree of much greater mass than the fruits produced in any given harvest season. But this inefficiency has been tolerated because of the tastiness of the product, the availability of suitable land, the durability of the apple factory over the life span of the tree, and the low price of sunlight and CO₂ used by the tree for energy and food.

As understanding of life and of its genes progressed, transgenes were in vogue for transferring a gene from one species to another. For example, tobacco plants were engineered to express foreign proteins for the advantage of cheap growing and established protocols for purification to useful (and animal free) protein products. And in other applications of genetic engineering, animals were transgenicly altered, for example to express a transgenic protein in their milk. In still other applications, bacterial and yeast transgenic cultures were engineered and maintained with restriction sites allowing engineers to predictably incorporate a transgene into the cultured cell.

Two common vaccines in use today, those against hepatitis B and human papilloma virus are produced in yeast factories, i.e., recombinant yeast organisms. While a valuable tool for protein production, such factories are susceptible to genetic drift as cultures mature, thus requiring periodic restart of cultures from frozen originator stores. These factories are also sensitive to changes in the water, food, and even piping supplying the vat and cleaning/disinfection operations, that alter the vat and supply line surfaces, and therefore require frequent revalidations to assure consistency of product.

In conventional protein/polypeptide manufacture, to establish a culture as a protein factory, expression cassettes containing the DNA sequence of interest, promotors, transcription factor recognition sites, etc., are inserted into the production cell and best producing populations are selected. Normally, the cell production factories will be isolated as single cells to produce monoculture which are expanded and assayed. Based on the assays several monocultures are selected for expansion and further testing. A select monoculture is then selected and propagated for fractionation into present and future growth stocks and freezing to preserve those not actively producing. Strains that are not selected for final production, but that were judged to be potentially good candidates are also frozen as insurance against a future finding requiring withdrawing the production strain from production.

Actual bio-production generally involves an initial spiking in a seed fermentation tank (vat) with either a small aliquot from a recent production run or a scheduled restart from frozen stock samples. The seed vat output is then used to spike the production vat, a tank about five to twenty times the volume. By selecting rapidly propagating cell factories that constitutively produce the target protein at a high rate about 10 grams of desiccated vaccine protein containing cells per liter production vat can be produced in each run—usually about once per week. The protein of interest is sometimes secreted into culture medium, but often remains in high concentration in the harvested cells. The desiccated product from cells, each having been engineered possibly with dozens of copies of the gene of interest, will be expected to have a significant fraction of its protein as the engineered protein. In general, a single such batch is expected to be sufficient for 50,000 adult vaccine doses. Accordingly, for example, since three to four vaccinations are required to establish reliable immunization, each production run would supply enough vaccine for about 15,000 individuals.

### Cell-Free Production Systems

Cultured bacteria and yeasts however are living organisms and therefore are susceptible to minor changes in culture conditions such as presence of trace metal ions, e.g., from water supply or culture feed changes, replaced parts, etc. Replacing a valve or pipe section, or changes in water supply have been observed to affect growth conditions, sometimes impacting the rate of production, but sometimes changing the output, for example, though altered glycosylation or protein folding. Though cell cultures remain a popular source of biomaterial (including vaccines), cell-free “cultures” are gaining prominence.

Generally, cell-free reaction mixtures for protein production include a cell extract (lysate), one or more energy sources, amino acids, sometimes added cofactors such as magnesium, to which DNA of interest is added. The extraction process preferably would have removed the source DNA and plasma membrane from the extracted cell, but will contain an unidentified mixture of components. Sometimes the extract is boosted with additional ribosomes, tRNA (possibly distributionally optimized for the DNA to be transcribed and translated) and supportive enzymes and factors (recognition, initiation, elongation, etc.). Energy sources vary, but often may include phosphoenol pyruvate, acetyl phosphate, and/or creatine phosphate. Although sequenced mammalian genomes have revealed several hundred copies of tRNA genes (some possibly tissue specific), the machines and methods of the present invention are operative when only at least one tRNA corresponding to each amino acid residue in the manufactured product is included.

The use of cell extracts provides a functional protein synthesis machinery, but is undefined, i.e., dependent on unknown factors of the cells used for extraction.

In response, a more defined system using engineered components has been marketed as PURESYSTEM™ (PURE) comprising “initiation factors (IF1, IF2, IF3), elongation factors (EF-Tu, EF-Ts, EF-G), release factors (RF1, RF2, RF3), ribosome recycling factors, 20 aminoacyl tRNA synthetases, methionyl tRNA formyltransferase and pyrophosphatase, all bearing a 6×His tag, ribosomes and tRNAs isolated from specially engineered E. coli strains, together with one or more nucleoside triphosphates (NTPs) and amino acids, an ATP-generating catalytic module and recombinant T7 RNA polymerase. The advantages of the PURE systems include reduced levels of contaminating proteases, nucleases, and phosphatases, greater reproducibility resulting from more defined chemistry, and the flexibility of a modular system.”

Polypeptide synthesis is energy-intensive. Generally, 4 ATP equivalents are required to form each peptide bond, e.g., 2 ATP equivalents for activation, 1 GTP to transfer charged aminoacyl-tRNA to the A site of the ribosome, and 1 GTP to process the ribosome through coupling to EF-G (also a GTPase).

Conventionally, cell-free protein synthesis has its output determined by adding a DNA template of interest which is transcribed by a recombinant phage T7 RNA polymerase to form the mRNA template that directs polymerization by the ribosomes. Supply of a stable mRNA content or periodic or continuous supplementation can remove the necessity for DNA and the cumbersome transcription support machinery. Limiting reaction pathways in the synthetic process allows for optimization of those remaining without constraints imposed to provide suitable conditions for those unnecessary additional pathways. Rates of synthesis, especially in the presence of stimulatory concentrations of polyamines, can be also increased by optimization of the 5′ untranslated region.

### Optimizing Polypeptide Production

Chemical energy is needed to drive these synthetic reactions. The enzymes used in the system rely on high energy phosphate compounds. Many options for providing intermediates for these energized phosphates are available, including, but not limited to: creatine phosphate, phosphoenolpyruvate, 3-phosphoglycerate, ATP, inorganic phosphate, etc. along with the enzymes appropriate for making the energy available to the reactions. Enzymes to equilibrate phosphorylation states of the nucleosides and to couple ATP/GTP regeneration are also advisable to enhance production. Aminoacyl amino acids can be incorporated in the mixture when desired. Control of GSH, Mg, Ca, Se, Fe, Cu, S and other cofactors in sufficient and optimal amounts may help maximize outputs.

One option is to provide a separate source of energy generated, for example, in at least one side tank. This embellishment allows for independent selection of optimal conditions, such as ionic strength, pH, temperature, Mg concentration, etc. so that the entire protein production process can be optimized by separately optimizing conditions in each component part. For example, ATP/GTP generation conditions when optimized may be at least about 2° C., 3° C., 4° C., 5° C., 7° C., 10° C., 12° C. different from other reaction component parts and may have conditions specifically optimized for that feeder function.

In a system where GTP is used for initiation by IF2 protein incorporated in ribosomes, the present process may use, for example, Nm23-H6, a particularly heat-stable kinase for interconversion of ATP-GTP. This example is not meant to suggest that ATP/GTP generation must be at a relatively elevated temperature only that conditions of various aspects might be individually optimized. At the discretion of the designer/operator, a portion, for example a reaction container comprising heat stable ribosomes may operate at a temperature in excess of that for optimized ATP/GTP generation. “Optimized” is not meant to imply maximum rates of production, merely a production protocol that is desired for whatever reason to be best for that system at that moment.

Ribosome selection is important for optimizing the in vitro synthetic machinery and desired performance. For a simple protein manufacturing task, plant cells, for example, may be selected as a ribosomal source. Harvesting ribosomes from lysed plant cells is rather straight-forward. The lysate is provided with an abundance of “tagged” mRNA. The tagging is any feature that facilitates harvesting, e.g., including, but not limited to: a binding moiety on the RNA strand itself, an encoded peptide sequence that is a target of, for example, coated glass beads, an RNA sequence complementary to a fishing sequence, etc. After allowing the mRNA to initiate synthesis and before termination, the RNA attached to the ribosome can drag the ribosome from the crude lysate. Similarly, a peptide sequence that binds a harvestable fishing molecule can drag the ribosome from the bulk. Photosynthetic plant cells have an advantage of carrying 3 populations of ribosome. While chloroplast ribosomes are generally most abundant and most active, the mitochondrial and cytosolic ribosomes are also present. Tagging the translated peptide allows selectivity based on differences in genetic codes recognized by the different ribosomes.

Mitochondrial ribosomes may be preferred in part because their library of tRNA is restricted. Chloroplasts tRNA libraries are larger than mitochondria but still limited with respect to nuclear DNA based ribosomes, but since mitochondrial genomes in many plants have imported chloroplast tRNA and have ability to use cytosolic tRNA, animal ribosomes may be preferred in some embodiments for the parsimonious use of codons. Several advantages of mitochondrial ribosomes may extend from their inclusion of significantly more proteins than other ribosomes, “more” may be counted as shear number of different proteins, but it may also be accounted as percent [Mitochondrial ribosomes can be 65 to 70% protein, whereas protein content in bacterial ribosomes is only about 33%.].

Identification of all or even any of the ribosome associated proteins is not a requirement for practicing the invention. Since different species will have different mitochondrial proteins encoded by the host genome, mitochondrial ribosomes from different species are expected to bring different properties. The ribosome(s) selected for use can be considered the relevant unit.

Mitochondrial ribosomes of several species have marked stability at elevated temperatures. This heat tolerance may result from adaptation to the massive amounts of heat generated in these highly active organelles. Mitochondria that produce significant amounts of ATP, an exothermic process, will increase their local temperatures within their host cell. Several plant chloroplasts share this property of significant heat generation.

Extremophilic bacteria have similar heat stability, however, many of these extremophiles are slow polymerizers, possibly because of lack of selective pressures from competitors in the extreme conditions. Thermophile sourced ribosomes are preferred in many processes because the increased temperature accelerates diffusion and kinetic chemical reactions. Additionally, many RNAses become denatured and non-functional as temperatures increase so maintain relatively high reaction temperatures can protect RNS from enzymatic degradation.

Ribosomes may be supplied as stabilized complexes, e.g., as embedded in phospholipid or other membrane structures or attached to membranes through one or more binding proteins. They may also be stabilized on a solid or semi-solid support. Any suitable stationary or confinable substrate may be used for solid support, e.g., a material having a rigid or semi-rigid surface including, but not limited to: a bead, a vessel wall, a liposomal vesical, a fiber (natural or artificial), a sheet, a mesh, a web, a semi-solid gel, etc. A solid support may be biological, non-biological, organic, inorganic, or a combination of any of these that may be configured as particles, strands, precipitates, gels, sheets, membranes, tubing, spheres, containers, capillaries, vesicles, pads, slices, films, plates, slides, etc. The substrate may be for example, a silicon or glass surface, a polymer—preferably an organic polymer, polycarbonate, (poly)tetrafluoroethylene, (poly)vinylidendifluoride, polystyrene, a charged membrane (possibly hydrophobic in a planar, spherical, semispherical, etc., shape), such as nylon 66 or nitrocellulose, a natural fiber, a silk, a protein, insect, silk, arachnid silk, etc. and/or combinations thereof. Solid supports may be used to fix or control location and/or activity of any up to all of bioactive elements of the machine. “Bioactive” is used here in a generic sense including, but not limited to: participating in or causing a biochemical reaction, stabilizing an affixed molecule, acting as a sink for controlling concentration, removing unneeded or harmful molecules, carrying a signal for monitoring synthetic processes, reporting pH or other relevant concentration, interacting with associated machine elements for controlling machine activity, carrying or delivering a bioactive element (including, but not limited to: ligand, reactant, product, activator, scavenger, reporter, etc.), shielding an element from a modulating signal (e.g., an inhibitor, electromagnetic wave, magnetic field, temperature gradient, chemical gradient, etc.)

A solid support may be employed in many manners. For example, the support may function simply to control the location (e.g., keep it from diffusing or from being washed off, to serve as a carrier for precise or timely delivery, to maintain exposure to other components of the synthetic factory, to provide tagging and/or visualization, etc.). In an elegant embodiment, a solid support may be structured as a fiber, e.g., a spider silk fiber that can, for example, store and deliver reactant, product, active ingredients, scavengers, etc. The fiber may be used to provide a large surface area for reactions. The fiber may be wound and unwound to expose its surface with attached substance(s) at selected times. In some embodiments, a base stock may be maintained stabilized on a wound fiber, rolled or folded sheet or mesh, etc. Attachment may be through covalent, ionic, hydrophobic, hydrogen binding, or other affinity. Attachment may be irreversible or randomly or controllably reversed.

The elevated protein content of mitochondrial ribosomes provides additional sites of protein-protein interactions between the ribosomal subunits allowing multiple interaction sites to coordinate maintaining a more stable structure. In view of observations that increased H⁺ bond strength from a shift to more C—G pair nucleotides in the ribosomal RNA fails to account for the comparatively higher ribosomal RNA melting point, the additional coordinating bonds involving ribosomal proteins are likely contributors to improved stability, including heat tolerance. Many advantages including, but not limited to: lower cost, accelerated reaction rates as diffusion rate increases [but tempered by genetic responses that may change catalytic efficiency], less dependence on the ambient temperature, denaturation of competing inhibitors, inactivation of contaminants, resistance to common infections, etc., attach to conducting biochemical reactions at relatively higher temperatures such as available using mitochondrial ribosomal material. Another advantage of mitochondrial ribosomes is the reduced reliance on 5′ caps or Shine-Delgano sequences, thereby allowing more straightforward sequence selection for mRNA when this is a factor.

For a tank based or batch system, the synthetic machinery includes multiple components, e.g., initiation factors (IF1, IF2, IF3), elongation factors (EF-Tu, EF-Ts, EF-G), release factors (RF1, RF2, RF3), ribosome recycling factors, a minimized number of aminoacyl tRNA synthetases dependent on the amino acids actually used, methionyl tRNA formyltransferase and pyrophosphatase, ribosomes from select source and tRNAs minimized to correspond to mRNA sequences to be translated, appropriate amino acids, an ATP/GTP-generating catalytic module and nucleoside-diphosphate kinase. The actual initiation factors and elongation factors in the composition will be those corresponding to the ribosomes and the ratios of tRNAs and amino acids in the mix will correspond to one or more mRNA molecules to be translated. The concentration of elongation factors may be increased or lessened if faster or slower synthetic rates are targeted. Elongation factors, when rate limiting, can be added to increase synthetic rates or decreased to slow synthetic rate, for example if temperature threatens system stability by denaturing one or more components. By eliminating unneeded or superfluous components the synthetic cycle time can be shortened, cost is reduced by more efficient production and less waste of unused substrate. Consumables are preferably added in process to maximize production rates and when desired, to control heat generated within the system or to cool or heat the synthetic module as necessary.

Nucleoside diphosphate (NDP) kinase are ubiquitously expressed and highly conserved across time and species. Multiple genes encode the NDK proteins in different cells and organelles. NDKs catalyze the transfer of the y-phosphate of triphosphate nucleotides to diphosphate nucleotides. The activity of NDK allows the in vitro stew to support high energy phosphate actions with only one nucleoside triphosphate fueling the protein synthesis.

An optional feature involves triphosphate generation attached through feed lines to the synthetic module. This allows carbon fuel generation of the energetic nucleosides to be separately controlled for maximal effect. Recycling of expended nucleoside mono phosphates may be accomplished through filtration or dialysis. Specific or non-specific macromolecules or sites of attachment for the synthesized protein(s) may be included to reduce inhibitory effects that the synthesis product(s) may produce. Some embodiments may benefit from inclusion of vesicles to secure nascent polypeptides.

Some embodiments may incorporate electromagnetic or magnetic sensors, actuators or stimulants. For example, magnetic fields may be used to induce movement of magnetized particles. Magnetic fields may assist in controlling location, including, but not limited to: suspension of particles, temporarily confining an active substance, removing component(s), harvesting product, etc.

Electromagnetic sensors may sense and/or transmit electromagnetic waves which may include visible or invisible light, including, but not limited to: the visual spectrum, infrared radiation, ultraviolet radiation, x-ray, radio waves, wi-fi signals, etc. Molecules, complexes or supports attached to elements in the synthetic assembly may fluoresce, be chemiluminescent, be luminous, be photochemically activated or inactivated, etc.

As an alternative to a tank/batch system, a flow-through and/or dialysis exchange system can permit control of concentrations of the substrates, enzymes, cofactors, etc. to be present ideally when necessary to minimize competitive or spatial interference with other reactions and thereby to optimize synthetic throughput. Ribosomes attached to solid phase act as synthetic machines. Substrate and fuel flow freely by each ribosome to supply the energy and substrate for peptide polymerization. mRNAs are continuously available and their lengths, sequences, optimal temperatures, mix of tRNA required, etc. can be updated while the process continues to run. The system is highly controlled since flow rates of various feed lines are adjusted according to sensors en-route and at the outputs. In a flow through system tRNA and/or amino acid, including modified amino acids, can be fed based on the optimal times need in the polymer chain. This can speed production by removing competitive interference from tRNA and/or amino acids not relevant at that stage of polymerization.

Carbon containing energy sources including, but not limited to: glucose, galactose, G6P, 3PG, acetyl phosphate, oxalic acid, phosphoenolpyruvate, starch, maltodextrin, etc. support peptide production in all systems. Polyphosphates can also be incorporated to act as phosphate donors. Enzymes appropriate for the substrates should also be included.

Oxygen free synthesis is possible when using glycolytic ATP production. Small molecular additions such as ATP molecules might be added to the synthesis through dialysis in a semi closed system or en-route in a flow through system.

A gas phase flow through system is also available using particulates suspended in a cloud. Additions are accomplished using vapor phase, misting, spraying, etc. Size, density, electrostatic interaction, magnetic forces, etc., may be used as factors or mechanisms for delivering or harvesting components. One particularly elegant of this embodiment would be its use in a zero-gravity environment such as in a spaceship or satellite.

Multiple proteins can be assembled in the same system. Chaperonins can be expressed from mRNA or added intact to such systems to improve desired outcomes when peptide folding is advantageous.

Amino acyl tRNA synthetases specific to the amino acid substrate and the tRNAs in the preparation will activate amino acids. The stoichiometric ratios of the amino acids are preferably optimized for the mRNA template(s) to be translated and polypeptide(s) to be produced. Ratios of proteins can be modulated by controlling the amount of each mRNA present with accounting for length and assembly times for each polymerization. The stoichiometric ratio may not precisely match the ratio of amino acids provided in the mix when presence of other interfering tRNA complexes may compete to impede the rate of reaction.

The nucleic acid template will generally be selected using a polypeptide as a model for ordering codons corresponding to each amino acid residue of the polypeptide. However, the invention is also applicable where the polypeptide is uncertain—perhaps due to uncertainty about the reading frame to be read at the ribosome, existence of overlapping reading frames, mutations altering reading frame, etc.—and a nucleic acid underlies the desire for polypeptide product. The corresponding nucleic acid template, mRNA or where appropriate in preparatory processes, DNA, should be optimally selected for the tRNA to be used. Ribosomes from a species of interest may be chosen for optimal activity at the temperature chosen for polymerization. A stop or pause in the polymerization process, e.g., to add a specific modified amino acid at a specific location can be accomplished by including in the code a tRNA complementary sequence and only adding the tRNA at the appropriate pause point.

Antioxidants may be advantageously added in circumstances where oxidative damage or oxidation side reactions interfere with desired results. The polyamines participate in protecting ribosomes from oxidative damage, e.g., by stimulatory binding to catalases.

tRNA can be re-engineered to match the associated amino acid to an unconventional codon and even to modified nucleic acid when stability is enhanced in this manner or for other design intentions.

Cell free production constituents may be optimized by, for example, selecting elongation factors (EF-Ts, EF-Tu, EF-G, and EF4), ribosome recycling factor (RRF), release factors (RF1, RF2, RF3), chaperones (e.g., DnaK/DnaJ/GrpE and/or GroEL/GroES), BSA, spermidine and tRNAs in ratios and temperature/pH optimal performances matching the temperatures, pH etc. conditions selected for synthesis. Polyamines, including, but not limited to: putrescine (PUT), NH₂(CH₂)₄NH₂; spermidine (SPD), NH₂(CH₂)₃NH(CH₂)₄NH₂; and spermine (SPM), NH₂(CH₂)₃NH(CH₂)₄NH(CH₂)₃NH₂ (in mammals) stabilize ribosomal structures and appear to decrease the optimal Mg⁺² concentration necessary for protein synthesis. Spermidine appears to control transient dissociation of ribosomal units. Ribosomes from different sources will have analogous ribosomal proteins and factors which the skilled artisan will similarly optimize.

Additional or alternative chaperones that may be used include but are not limited to: Hsp60s (inclusive of Hsp60A, Hsp60B, Hsp60C and Hsp60D), Hsp10, Hsp40, Cpn60, mt-Cpn60, Cpn10, mt-Cpn10, ch-Cpn20, ch-Cpn60, HSP65, Clp, etc., gene expression products of BBS10, BBS12, TCP1, CCT2, CCT3, CCT4, CCT5, CCT6a, CCT6b, CCT7, CCT8, CLP8, HSPD1, HSPD1 HSPE1, MKKS, etc.

Spermine synthase which interconverts spermine to spermidine is useful for maintaining proper balance of the polyamines. Decarboxylated S-adenosylmethionine is a useful supplement to support putricine conversions to other polyamines. Spermine oxidase (SMO) and acetylpolyamine oxidase (APAO), which convert spermine into spermidine and 3-aminopropanal, and N1-acetylspermine into spermidine and N-acetyl-3-aminopropanal, respectfully are optional additions. Polyamines and their associated enzymatic systems are chosen with respect to the ribosome source. Optimization of polyamines for specific conditions such as chosen temperatures, ionic strength and pH will improve synthetic outcomes.

Synthetic assembly devices are preferably modular to allow responsive optimization when a new synesthetic need is identified. Distinct modules may be incorporated as preparations prepared to be dissolved, special solvents, ion preparations, heat stable enzymes, carriers, chaperones, etc., selected from a menu to optimize the planned production run. Modules may be formatted as strap on elements, e.g., separate reaction cassettes for each desired protein or a compatible assortment of proteins. Strap on elements may be, for example, water purifiers and/or filters; ligand or substrate production and/or delivery modules, e.g., for synthesizing and delivering energized phosphate compounds; amino acids; carriers; lipids; reporter compounds; scavengers; cleaning plant(s); harvesting devices; etc.

Many of the known benefits of the polyamines in living organisms will not be relevant or will have decreased relevance in in vitro biologic production. For example, although, chemically polyamines offer protection from reactive oxygen species as evidenced in bacteria, yeast, and mammalian cells, e.g., as free radical scavengers, as quenchers of singlet oxygen, and as general free radical and active oxygen scavengers, their compounded supportive properties of stimulating the synthesis of anti-oxidative and stress proteins such as superoxide dismutase, heat shock proteins, and cell cycle regulators by activating transcription of these in the nucleus will not be relevant in vitro.

Activities, such as control of ion fluxes and several modulatory effects in metabolic pathways indicate that polyamines in general, e.g., from species that differ from the ribosomal species may be used to dampen or hasten reaction rates. While a spermidine/ribosome ratio of about 1:1 may prove adequate for the synthetic system to function, ratios of about 3:1, 5:1, 7:1, 10:1, 12:1, 15:1, 20:1 and even 25:1 or higher may be selected, especially for longer batch runs where effective polyamine levels may be consumed during operations. Polyamines may be one of the groups of substances supplemented during production.

A first candidate for protein to be included in vaccine is any surface protein(s) of a pathogenic virus. However, since viruses replicate so prodigiously and are constantly being attacked by immune systems the surface proteins often vary significantly with viral strain or subtype. Thus in conventional practice, identifying a surface protein, engineering the DNA into an organism, growing the organism to produce the surface protein may produce ineffective product because many new infections will comprise different surface proteins. A less complex production allowing shortened time from viral characterization to vaccine production can be more effective. Also the simpler production methods will allow more rapid switching of outputs to match those of modified but related surface proteins. But since the surface proteins are the virus' gateway into a host cell, the surface proteins must at least retain ability to bind and infect a host. Inhibiting such interactions is the function of one alternate, non-vaccine embodiment of this invention.

A secondary vaccine target is a vaccine that induces immune response against viral particles expressed and appearing in the plasma membrane of an infected host cell. DNA vaccines are one attempt at his approach. A complicating factor lies in a host organism's immune response against such virally infected cells. Sometimes, the auto-immune response characterizes the disease. For example, hepatitis B does not significantly impair performance of the infected liver cells. However, in unresolved hepatitis b infections, the continued immune attack on host liver cells not only acts to eliminate infected cells, but the cytokines and oxidants released in the vicinity of infected cells removes many healthy cells from the viable pool. Since the virus itself is not attacked by the immune system there is less selective pressure to force viral change so these types of infection can permit stable surface proteins and thus persistence in surface protein expression.

When selecting peptide spans for antigenic presentation, taking such preference into account when choosing core amino acid residues may improve selection efficiency and help minimize wasted trial effort. Restricting initial selection of potential antigenic targets to fit these formats can accelerate the development process. Wild type pathogenic protein messenger RNA can be used in the invention. But modified RNAs can also be efficiently produced. Different MRNs of different lengths can be simultaneously produced. To achieve target ratios of proteins the number of mRNA copies for each protein can be adjusted to account for synthesis time—based possibly on length, inclusion of specific amino acids, and presence of amino acids or tRNA. Temperature, specific amino acids, concentration or phosphorylation of any of the enzyme factors, ionic strength, divalent cations, flow rates in flow through systems, rate of ATP/GTP feed, etc., in essence any component that can be rate limiting can be metered for desired production rates.

Immune responses to foreign protein products may pose problems for both recipient safety and product efficacy. Immunologically based adverse events, such as anaphylaxis, cytokine release syndrome, and cross-reactive neutralization of endogenous proteins mediating critical functions, are rare, but have caused sponsors to terminate the development of what may otherwise have presented efficacious results. Untimely and ineffective immune responses to vaccines will neutralize immunogenic stimulation against the target pathogen. Proteins eliciting these responses may signal cross reactivity with another pathogen's proteins.

Because most of the adverse effects resulting from elicitation of an immune response to a foreign protein appear to be mediated by humoral mechanisms, a test for circulating antibody to each of the vaccine proteins can avoid inclusion of non-efficacious proteins in the candidate vaccine.

When chicken eggs or other biological factories are used to culture the viruses to be killed and extracted, components from the host can induce reaction, such as anaphylaxis. Allergies to hosts are thus contraindicative against vaccination in these people. Using a non-cellular system can almost totally eliminate these issues.

When the source of materials for manufacturing the proteins is human, for example cultured human cells or human mitochondria, concerns of allergic response to the protein element and other living organism derived components that may remain in small quantities in the vaccine are effectively eliminated. Similarly, deriving synthetic machinery from human microbiome species, e.g., human E. coli virtually eliminates concerns of allergic response from the manufacturing process.

Instead of the historical, arbitrary, as is, system with killed or attenuated viruses, molecular engineering has provided optimization tools allowing for example, attenuation to be accomplished by gene elimination or substitution, by inserting specific sequences easily testable before batch release, engineering virus for killed vaccines to hyper produce or non-produce select proteins—to grow especially rapidly in a paired host cell—to tolerate optimal growth conditions, etc. Taking this one step further, the present cell-free vaccine synthetic system can be modified to produce any protein(s) simply by stocking appropriate mRNA. The mRNA(s) can include tagging sequences for facile harvesting and cleavage sequences responsive to a desired protease. One or more tagging sequence(s) can be incorporated in any production run. Each tag will be recognized and specifically bound by an immobilized substance or free substance that will allow discrimination of the tagged protein of interest from other molecules. The tag encoded by each mRNA may be exclusive to that polypeptide in its specific run or may be shared with one or more, including all, peptides being synthesized in the run. Preferably the tag incorporates a cleavage site whereby chemical or enzymatic cleavage can cleave the synthetic polypeptide from most or all of the tagging ligature. Length can match the wild type protein, or may be fragmented of chained to produce any length of polypeptide. Since proteins are presented as fragments for immune recognition, lengths of presentable lengths can be walked along the wild type or modified sequence to produce a collection of polypeptides in ratios desired. Or the tagging/harvesting process may allow specific fractions to be harvested for lesser proportion or greater proportion in the packaged protein composition. As mentioned above, undesired sequences, such as those cross reactive with common pathogens or to host protein portions.

After selecting at least one polypeptide for manufacture, perhaps as an antibiotic compound, a hormone or pseudo-hormone, inclusion in vaccine composition, a corresponding mRNA template designed to instruct a ribosome to generate the polypeptide sequence. The mRNA may be optimized for an existing or preferred production process with its tRNAs and other components readily available or the constituent mixture for producing the polypeptide(s) may be correspondingly optimized for production of the select product(s). In some circumstances a polypeptide may not be known, the process only requiring a mRNA template. The mRNA template(s) may be in frame or out of frame of the normal expression product or may encode multiple expression products. The present invention is also applicable in these circumstances.

The packaged protein mixture may be lyophilized or otherwise desiccated, may be stored as a liquid solution, may be organized as a gel, may comprise single polypeptide sequence or may comprise multiple sequences, for example, 1, 2, 3, 4, 5, 6, 7, 8, 9, 10, 11, 12, 13, 14, 15, 16, 17, 18, 19, 20, 21, 22, 23, 24, 25 or more different polypeptide sequences in any desired ratio between any two polypeptides, for example the ratio of one polypeptide to a reference may be 1, 2, 3, 4, 5, 6, 7, 8, 9, 10, 11, 12, 13, 14, 15, 16, 17, 18, 19, 20, 21, 22, 23, 24, 25 or more. The reference polypeptide can be any polypeptide in the system, including polypeptides manufactured or not manufactured during that run, for example, an enzyme in the system, a spiked reference polypeptide, a non-peptide reference molecule or substance, etc. Standard excipients can be selected for compatibility with the active ingredients and any incorporated carriers. However, no FDA approvable substance should be ruled out for possible stabilization, viscosity maintenance, preservative effect, or any other advantageous purpose.

As manifested in the 2009 flu vaccine administration history, vaccines occasionally induce auto-immune disease. Some versions of the Pandemrix 2009 flu shot contained a protein portion that mimicked the structure of receptor in the human brain that binds to a hormone called hypocretin. This hormone is active in the reticular activing system for keeping people awake. People with narcolepsy have lower levels or lower effects of hypocretin, which led the researchers to hypothesize that problems with hypocretin or its receptor could play a role in the sleep disorder. Researchers who analyzed blood samples from 20 people in Finland who developed narcolepsy after a Pandemrix vaccination found that these people had antibodies that bound not only to the H1N1 flu virus of the vaccine but also to their hypocretin receptors.

Artificial Intelligence/big data can be used to screen candidate vaccine proteins for portions complementary to or matching genetic sequences. The entire protein with mimic portion(s) might be eschewed; alternatively, mRNA encoding the protein could be made without the mimic portion.

Pre-manufacture application of Artificial Intelligence and its growing capabilities regarding patterns recognitions is a proven tool that we can now apply to antigen selection. Specifying inclusion of considerations relating to avoiding in vaccine sequences that match host fragments with similar sequence pattern will significantly reduce time from initial disease organism identification to the production of effective vaccine. Such in silico screening in human vaccine candidates can immensely shorten the time from viral identification/sequencing to delivery of effective vaccine to at risk locations.

Additional constraints in Al instructions, for example, limiting the number of amino acids required, stability of the product for shipping/storage, etc., cost of components, cross reactivity with other pathogenic organisms, etc., can be used to improve product utility, ease of product and costs.

Stores of polypeptides may be prepared and disbursed to depots for ready availability in case of sudden demand. Stores may comprise base stocks or essentially complete compositions. When stored as base stock, potions of said base stock may be released for a quick response while other portions are retained for augmentation with additional components as they are identified and produced for incorporation. Said stores may comprise proteins of any sort, for example, vaccine proteins, treatment proteins, cleaning proteins (enzymes for disaster cleanup), herbicide, pesticide, anti-viral, anti-bacterial, receptor blocking, enzyme inhibitor, precipitant, carrier, etc. As an example, a base stock flu or other disease vaccine may be prepositioned for vaccination on a seasonal basis. The stock could comprise several vaccine proteins that may be ready for the coming flu or other disease season or eruption. Base stocks may be mixed or matched, delivered as the archived base, used to expeditiously synthesize new product, maintained in storage as a deterrent against bio-weaponry or terrorism, etc.

But then when a new strain develops or becomes more prevalent, its genome can be expeditiously sequenced, determined as sense or anti-sense, screened for likely epitope regions and autoimmune risk regions. Following this, corresponding mRNA might be chemically polymerized or might be grown in a biological factory such as a cell or virus. The new mRNA is then fed into the high-throughput ribosomal factory.

The ribosomal factories can be prepared as kits with optional items or items to be provided in mRNA specific ratios available for addition in the desired quantities. The kit format allows flexible mass manufacturing of a protein or polypeptide of desire in rapid response to a newly discovered need, a newly identified target, a new market, etc.

The product can be retrieved from storage or transit and prepared for administration where needed, for example to fish in a tank or farm, as a vaccine, for spraying on a plant, field, lake, etc. The transit or stored composition may be ready-made (No or minimal preparation necessary to prepare for distribution) or may require solubilization (for example if lyophilized or surface coated), aliquotting, coating or gel formation, metered dosing, etc., for distribution to recipient. In the example of a flu vaccine, several flu proteins may be predistributed and pulled from storage when the season is about to stimulate demand. A new flu on the horizon may have components thereof prepared and added to a prepared base. The base may have been desiccated and the addition may be shipped in liquid form with a formulation to solubilize the prepared base contents. The prepared base contents may already include sugars, amino acids, enzymes or other components to be consumed or used in any specific distribution event. The solubilized product is then labeled for distribution to the recipient and administered in an appropriate manner, for example as an injection of a flu vaccine.

Synthesis of polypeptides has been a domain of life forms for eons. Much more recently mankind has developed methods, for example, solid phase synthesis that can chemically produce short peptide strands, some of which may be of molecular structure suitable to act antigenically. However, solid phase synthesis is rather cumbersome in speed and in amount of product produced. Error rate also is unacceptable for many purposes and drastically increases with length of the polypeptide molecule. Adapting a cell-free synthetic system to this purpose solves several of these problems.

Speed can be increased by incorporating ribosomes selected for rapid polymerization. Mitochondrial ribosomes are one example of simplified structures capable of operating at high speed and low error rate. Many mitochondrial ribosomes are stable across broad temperature ranges allowing polymerization to be ramped up with temperature increases. The cell-free system removes restraints on mitochondrial source. Yeast, plant, animal, even human mitochondria may be selected as the source of ribosome. Ionic strength can be modified to modulate macromolecular binding as temperatures are modulated. Inclusion of stimulatory or stabilizing proteins e.g., elF5a, can result in more rapid protein synthesis.

Where appropriate, in vitro glycosylation can be performed during synthesis or during post synthetic processing. As proteins are synthesized in the endoplasmic reticulum, secondary structure (folding) exposes and hides different parts of the nascent polypeptide. Inclusion of selected glycosidic enzymes with relevant glycosides, is an option available in this in vitro synthetic machine and process. In normal cellular protein synthesis, Glycosylation is accomplished in specific regions of the cell with dedicated glycosylation enzymes. Different glycosylation patterns ensue depending on trafficking of proteins within the cell. Presence of the enzymes and active sugars may provide sufficient glycosylation patterns for the protein's intended use. ThermoFisher Scientific™ and CustomBiotech™ (Roche™) are two suppliers of glycosylation tools and kits for in vitro use.

Match between ribosome and tRNA is not normally a serious impediment to peptide/protein production, but in a select system, reaction rates and conformity to the select sequence(s) for production, optimal tRNAs and mRNAs can be selected for the paired ribosome and the reaction conditions such as temperature, ionic strength, compatibility with other system components, insensitivity to aberrations, such as trace metals or other contaminants, resistance to chemical sterilization or antibiotics and the like. Polyribosomes in general appear more tolerant to elevated ionic strength solution with continued functionality.

Such cell-free production takes advantage of the evolutionary efforts of trillions of organisms to arrive at efficient methods of protein/peptide production while avoiding much of the cellular baggage. The system is thus robust in abilities, simple in design and reproducibility and low in cost.

Each amino acid is encoded by one or several triplets of bases (codons), e.g. UUU or UUC for the amino acid, phenylalanine, termination of translation by the triplets UAG, UAA or UGA and initiation of translation mainly by AUG, also encoding the amino acid methionine. The mRNA sequence is decoded starting from an AUG codon, followed by a sequence of codons, specifying the order of insertion of amino acids in the nascent protein, which is followed by a termination codon, signaling that the protein is to be dissociated from the ribosome for collection and packaging or processing such as enzymatic cleavage(s) and folding into a desired state. The link between the DNA and mRNA involves sequence recognition, bonding, polymerization, processing, transport, etc. These will not be necessary when mRNA is used as the production template. RNA modified for stability, but still complementary to the tRNAs that deliver the amino acids remove the complication of DNA transcription from eventual protein/peptide production.

In the bacterial cell there are about 50 different types of tRNA molecules, each composed of about 75 nucleotides. A cell-free system can select mRNA to eliminate the requirement for this redundancy. Mitochondrial and chloroplast ribosomes can be used similarly with their associated tRNAs selected to reduce the number of constituents in the brew.

The bacterial ribosome generally has three binding sites for tRNA, the A (aminoacyl) site, the P (peptidyl) site and the E (exit) site, formed in the inter subunit interface. mRNA binds in a track in the 30S subunit, through which it steps one codon at the time and one amino acid residue at a time, for polymerizing the desired protein.

As a more precise example, in bacteria, synthesis of a protein, following instruction of the mRNA commences with mRNA binding ribosomal 30S then an initiator tRNA, carrying formylated methionine, uses the P site for reaction facilitated by initiation factors 1 (IF1), 2 (IF2, a GTPase) and 3 (IF3). IF3 structure and activity is conserved across classes and species; bacterial IF3 is functional in bovine cell extracts. Eukaryotes and mitochondria have similar factors for assembly of the complex.

IF2 is instrumental in forming the GTP/formylated methionine-tRNA/30S ribosomal cluster. The GTP may originate from any source capable of delivering a triphosphate nucleoside when appropriate enzymes to ensure adequate transfer of the high energy phosphates to GTPs are active. Then the 50S subunit complex docks to the pre-initiation 30S subunit initiation prepped complex in an initiation factor aided reaction to form a 70S ribosome complex with the mRNA aligned for appropriate reading frame. Elongation can then commence.

In the elongation phase, aminoacyl-tRNAs enter the A site under guidance from a protein elongation factor Tu (EF-Tu, a GTPase) where GTP is hydrolyzed on EF-Tu followed by aminoacyl-tRNA accommodation in the A site to form the peptide bond catalyzed in the peptidyl-transfer center (PTC) of the 50S subunit. Elongation continues. EF-1 is considered the eukaryotic equivalent to EF-Tu.

For termination, when stop codons are read by class-1 release factors 1 (RF1, codons UAA, UAG) and 2 (RF2, codons UAA, UGA) hydrolysis of the ester bond linking a finished protein chain with the P-site bound tRNA results. This allows rapid release of the protein. This process elicits rapid dissociation of the class-1 release factors by the class-2 release factor 3 (RF3, another GTPase). The ribosome becomes available for to re-initiation with a next mRNA preferably controlled by ribosomal recycling factors (e.g., RRF) and by EF-G. Or in cases where spatial physics permit, multiple ribosomes can simultaneously act on a single mRNA strand in a staggered set-up to simultaneously elongate multiple peptide strands from the one mRNA strand. Simultaneous initiation and elongation are not incompatible with each other and do not require a termination before each can operate.

The polypeptide/protein synthesis system is not beholden to any special source(s) of component materials. While bacteria of any strain might be harvested to provide synthesis system components, any life form polypeptide/protein synthesis system can provide one or more components of the system. For example, mitochondrial protein synthesis systems are similar to those of the bacteria. The translational segment of the synthesis cycle has the four universal steps of initiation, elongation, termination and recycling for next initiation. Many aspects are conserved from the mitochondria through the bacteria. Notably, mitochondrial ribosomes have a higher protein content and thermal stability. Ribosomes may be selected from a set of mitochondria or perhaps archaea, while energy generation systems or may be plant, animal or even mixed sourced.

Also, if mitochondrial ribosomes are used, account must be made for the mitochondrial genetic code. The mitochondrial tRNAs will correspond to the code variances. Where systems favor two different tRNAs for methionine (an initiator tRNA-Met and an elongator tRNA-Met) sufficient balance of these and the tRNAs for other amino acids—taking the ratios of their frequency of use in the relevant production sequence is adjusted to optimize polymerization. When mitochondrial ribosomes serve as synthesis centers, the pre-sequences for transfer across the mitochondrial or chloroplast membranes are optionally omitted, though they may be retained for tagging or other purposes.

Another difference between mitochondrial and bacterial translation manifests in the translational factors guiding the process, especially initiation factors. In bacteria, we find three universally present initiation factors (IFs), IF1, IF2, and IF3. Mitochondrial IF2 (mIF2) is universally present, mIF3 is near-universal, with a handful of exceptions, and mitochondrial IF1 is universally lacking. Also a large group of lineage specific mitochondrial translational activators, the majority of which have been identified in the yeast Saccharomyces cerevisiae can be found in mitochondrial ribosome units. These are synthesized by the nuclear genome coding but transported to mitochondrion.

### EXAMPLES

### Example 1—Oil Mite

Oil tar sands workers near Vancouver develop hives, then fever. About one third of the fevers progress to rigor mortis like conditions with tetanic cramps throughout skeletal muscle. Several workers are placed on ventilators. An unidentified toxin is suspected. A criminal investigation rules out illegal drug use as the cause of this new malady. A toxin related to tar sands extraction methods is suspected. Comparison to industrial samples obtained at various stages in drilling/production from other extraction fields does not reveal a difference that might be responsible for the incapacity of these workers. Soil samples are taken to assess for possible causes. Although the sampling technicians are provided and diligently use sub HEPA size protective gear and positive ventilation garb, four days later two technicians who carpool together to the site sport the telltale rash and are prophylactically hospitalized. Three days later one technician ceases eating and becomes feverish. Her nurse develops the telltale rash. The nurse never had contact with the site, the protective gear or clothing from the technicians. The only possible exposure seemed to be contagion from airborne or body contact. It was discounted that this nurse had contact with bodily fluids. The aide most likely to have contacted fluids developed no symptoms. Then the other technician's girlfriend who had driven the truck back to base is staying in hospital. As the female technician approaches rigor, the girlfriend sports the rash, though both technicians had been segregated in isolation. A forensic disease technician samples fabric, mats and air from the truck and several other vehicles on site. No toxins are noted, but a common mite is found. Several vehicles and the portable breakrooms and toilet facilities (RV style) are vacuumed to collect live arthropods and their remains. These are worked up on the theory that a zoonotic disease might be operational.

A DNA virus is identified; and both technicians, but not the girlfriend test positive for the viral DNA in their blood and cutaneous biopsies. The viral genome is sequenced and all possible reading frames are processes as messenger RNA. The disease—being probably zoonotic and involving distant species, suggests a fungal mitochondrion based protein synthesis system to be used as the basis for synthetic machinery. Since arthropods and mammals are widely separated on the taxonomic charts, a closest similarity of mitochondrial ribosome and related tRNAs, factors and controls are made available for synthesizing 35 encoded protein sequences in all reading frames. Concurrently the sequences of these proteins are compared using international databases to human proteins and all nucleic acids. All sites in the area are treated with insecticides to eliminate the infected arthropods. Several available algorithms are used to select polypeptides with likely excellent immunogenicity in humans. A composite sampling of blood from twenty-seven of the thirty one workers known to have presented symptoms is reactive to seventeen of the hundred or so proteins presented. Six are highly reactive to these antibodies. These sequences are compared with the available human genomes for potential auto-recognition. As a back-up the other eleven are similarly assessed. Likely mimicry having been discounted, the mitochondrial RNA and ribosomal systems are commandeered to batch produce all six polypeptides. Three of the longer proteins with in excess of 180 amino acid residues are synthesized as intact molecules but also produced in 60 amino acid length fragments walked at 15 residue steps.

These polypeptides are injected into 4 strains of mouse and one canine with no apparent ill effect. Three volunteers from the region, two tar sands workers, one of whom was merely clerical with scant physical presence at the site, and a pipeline worker, act as phase 1 safety screeners, each receiving 35 μg protein in a single intra-muscular injection. With no deleterious results apparent, a small dose is injected IM into one suffering worker. No allergic reaction was noted. Others then are vaccinated. Three weeks to three months later samples obtained from those vaccinated reveal that seventy-three percent have detectable antibody following only one dose. All recipients are offered a second vaccination at their discretion. Vials with material sufficient to produce ˜2500 vaccine doses are stored in −180° freezers. A panel is studying recommendations for possible vaccination of all area workers and to plan responses for any similar recurrence.

1. An improved machine for bio-synthesizing polypeptides in a cell-free apparatus wherein at least one improvement is selected from the group consisting of: eliminating transcription as a step in protein production; avoiding addition of RNA polymerase; providing high energy phosphates in controlled feed amounts from an auxiliary production source; attaching ribosomes to a solid support; with a result that a flow through continuous peptide/protein production is possible; optimizing production through use of a restricted set of codons and associated tRNAs; selecting ribosomes from organelles or organisms to allow rapid synthetic processing and to lessen pathogenic danger.

2. The machine of claim 1 comprising a bioactive element affixed to a solid support.

3. The machine of claim 2 wherein said solid support is configured in a format selected from the group consisting of: particles, strands, precipitates, gels, sheets, tubings, spheres, membranes, vesicles, containers, capillaries, pads, slices, films, plates and slides.

4. The machine of claim 2 wherein said solid support comprises a composition selected from the group consisting of: silicon, glass, polycarbonate, (poly)tetrafluoroethylene, (poly)vinylidendifluoride, polystyrene, a charged hydrophobic membrane, nylon, a natural fiber, a silkworm silk, a protein, insect, silk and arachnid silk.

5. The machine of claim 1 configured to incorporate at least one changeable module.

6. The machine of claim 5 comprising at least one module comprising suspended particles.

7. The machine of claim 6 wherein said suspended particles are dispersed within a gaseous phase.

8. The machine of claim 1 wherein mRNA in said machine encodes at least one antigenic and immunogenic peptide.

9. The machine of claim 1 comprising mRNA designed in accordance with an artificial intelligence induced algorithm.

10. The machine of claim 1 comprising at least one chaperone protein.

11. The machine of claim 10 wherein said at least one chaperone protein is selected from the group consisting of: DnaK/DnaJ/GrpE, GroEL, GroES, Hsp60s, Hsp10, Hsp40, Cpn60, mt-Cpn60, Cpn10, mt-Cpn10, ch-Cpn20, ch-Cpn60, HSP65 and Clp.

12. The machine of claim 11 comprising at least one Hsp60 selected from the group consisting of: Hsp60A, Hsp60B, Hsp60C and Hsp60D.

13. The machine of claim 10 wherein said at least one chaperone protein is a gene expression product selected from the group consisting of: BBS10, BBS12, TCP1, CCT2, CCT3, CCT4, CCT5, CCT6a, CCT6b, CCT7, CCT8, CLP8, HSPD1, HSPE1, CD74, calnexin, calreticulin, and MKKS.

14. The machine of claim 1, further comprising optimizing content of an ion comprising an element selected from the group consisting of: Mg, Ca, Se, Fe, Cu and S.

15. The machine of claim 1, wherein an ATP/GTP generator operates with a temperature difference of at least 2° C. compared to a portion of said machine with said solid support attached ribosomes.

16. The machine of claim 15 wherein said temperature difference is selected from the group consisting of at least: 2° C., 3° C., 4° C., 5° C., 7° C., 10° and 12° C.

17. The machine of claim 1 comprising at least one: ribosome, tRNA, recognition factor, initiation factor, elongation factor, chemical energy source, RRF, aminoacyl tRNA synthetase, methionyl tRNA formyltransferase, pyrophosphatase, amino acid, ATP-generating catalytic module and RNA polymerase.

18. The machine of claim 17 wherein the at least one chemical energy source is selected from the group consisting of: phosphoenol pyruvate, acetyl phosphate, creatine phosphate, 3-phospho-glycerate, inorganic phosphate, and nucleoside triphosphate.

19. The machine of claim 18 wherein a nucleoside triphosphate is selected from the group consisting of: ATP and GTP.

20. The machine of claim 17 wherein the at least one initiation factor is selected from the group consisting of: IF1, IF2 and IF3.

21. The machine of claim 17 wherein the at least one elongation factor is selected from the group consisting of: EF-Tu, EF-Ts, EF4 and EF-G.

22. The machine of claim 17 wherein the at least one release factor is selected from the group consisting of: RF1, RF2 and RF3.

23. The machine of claim 17 wherein the at least one RNA polymerase comprisesT7 RNA polymerase.

24. The machine of claim 17 comprising at least one anti-oxidant.

25. The machine of claim 24 wherein said at least one antioxidant is selected from the group consisting of: superoxide dismutase, melatonin, ascorbic acid, glutathione, lipoic acid, uric acid, carotene, tocopherol, ubiquinol, DnaK/DnaJ/GrpE, GroEL/GroES and n-acetyl cysteine.

26. The machine of claim 17 comprising at least one component selected from the group consisting of: an ion or complex comprising an element selected from the group consisting of: Mg, Ca, Se, Fe, Cu, S and Fe; Nm23-H6, nucleoside diphosphate kinase, chaperone, spermine synthase, spermine oxidase, 3-aminopropanal, N1-acetylspermine, N-acetyl-3-aminopropanal and polyamine.

27. The machine of claim 26 wherein said chaperone is selected from the group consisting of: a gene expression product selected from the group consisting of: BBS10, BBS12, TCP1, CCT2, CCT3, CCT4, CCT5, CCT6a, CCT6b, CCT7, CCT8, CLP8, HSPD1, HSPE1, CD74, calnexin, calreticulin, and MKKS.

28. The machine of claim 26 wherein said polyamine is selected from the group consisting of: including, but not limited to: putrescine (PUT), NH₂(CH₂)₄NH₂; spermidine (SPD), NH₂(CH₂)₃NH(CH₂)₄NH₂; spermine (SPM) and NH₂(CH₂)₃NH(CH₂)₄NH(CH₂)₃NH₂.

29. A method for in vitro production of protein product, said method comprising operating a ribosomal based peptide synthesizer, said synthesizer comprising one or more ribosomes comprising rRNA and protein; initiation factors; elongation factors; release factors; ribosome recycling factors; a sufficient number and variety of aminoacyl tRNA synthetases dependent on the amino acids actually used; a sufficient number and variety of amino acids dependent on the amino acids actually used; at least one mRNA; methionyl tRNA formyltransferase; an ATP/GTP generator; nucleoside diphosphate kinase; and pyrophosphatase.

30. The method of claim 29 wherein the tRNAs are minimized to correspond to mRNA sequences to be translated.

31. The method of claim 29 wherein the aminoacyl tRNA synthetases are minimized to correspond to mRNA sequences to be translated.

32. The method of claim 29 wherein ribosomes are immobilized on a solid or semisolid substrate and a reaction mixture is flowed past the immobilized ribosomes.

33. The method of claim 29 wherein the protein product is an intermediate for vaccine.

34. The method of claim 29 wherein the synthesizer comprises multiple mRNA templates of differing sequences.

35. The method of claim 29 wherein the mRNA is assessed to eliminate sequences endogenous to the intended recipient.

36. The method of claim 29 wherein said synthesizer comprises at least one polyamine.

37. The method of claim 36 wherein said at least one polyamine is selected from the group consisting of: putricine, spermine and spermididine.

38. The method of claim 37 wherein a spermidine/ribosome ratio is selected from the group consisting of about: 3:1, 5:1, 7:1, 10:1, 12:1, 15:1, 20:1 and 25:1.

39. The method of claim 26 wherein a number of multiple mRNA templates is selected from the group consisting of at least: 2, 3, 4, 5, 6, 7, 8, 9, 10, 11, 12, 15, 18, 20 and 25.

40. The method of claim 29 wherein the algorithm seeks to optimize one or more factors selected from the group consisting of: immunogenic potential, cost of production, shelf life, biological half-life, solubility, viscosity effect, potential for induction of auto-immune response, protein-protein interactions, three-dimensional stability, cross reactivity and synthetic rates and accuracy.

41. The method of claim 29 wherein said one or more ribosomes are of human origin.

42. The method of claim 29 wherein said one or more ribosomes are of mitochondrial origin.

43. The method of claim 29 wherein said one or more ribosomes are of human microbiome origin.

44. The method of claim 29 wherein said one or more ribosomes are of E. coli origin.
